(19) United States
(12) Patent Application Publication
(10) Pub. No.: US 2025/0091886 A1
(43) Pub. Date: Mar. 20, 2025

(54) ZEOLITE BODIES

(71) Applicant: Cambridge Enterprise Limited, Cambridge Cambridgeshire (GB)

(72) Inventors: Lik Hong Wee, Kuching, Sarawak (MY); David Fairen-Jimenez, Cambridge Cambridgeshire (GB)

(73) Assignee: Cambridge Enterprise Limited, Cambridge Cambridgeshire (GB)

(21) Appl. No.: 18/580,336

(22) PCT Filed: Jul. 20, 2022
(86) PCT No.: PCT/EP2022/070417

§ 371 Date: Jan. 18, 2024

(30) Foreign Application Priority Data
Jul. 20, 2021 (GB) 2110421.1

(51) Int. Cl. C01B 39/40

(52) U.S. Cl. CPC C01B 39/40; C01P 2002/72; C01P 2004/01; C01P 2004/62; C01P 2004/64; C01P 2006/10; C01P 2006/12; C01P 2006/14

(57) ABSTRACT

Described herein are zeolite bodies, in particular those having improved physical and chemical properties, methods of manufacturing the zeolite bodies, and uses of the zeolite bodies, in particular in catalysis and gas separation.

### FIELD OF THE INVENTION

The present invention relates to zeolite bodies, in particular those having improved physical and chemical properties, methods of manufacturing said zeolite bodies, and uses of said zeolite bodies, in particular in catalysis and gas separation.

### BACKGROUND OF THE INVENTION

Zeolites are typically synthesised in fine (e.g. <10 microns diameter) powder form. Zeolites are widely used as heterogeneous solid catalysts and as support media for other catalytic materials. When used as such, typically in “packed beds”, the fine zeolite powders are typically shaped into larger bodies, such as granules having a maximum internal diameter of greater than 100 microns or greater than 200 microns or greater than 500 microns or even multi-millimetre sized bodies such as extrudates. Industrially relevant zeolite bodies, such as those used in catalytic processes, are typically multi-millimetre in scale. This shaping of zeolite powders improves handleability and helps to avoid excessive pressure drops and problems such as “channeling” that may often occur when fine powders are used. The use of larger zeolite bodies improves fluid flow, which serves to improve both mass and heat transfer properties under typical industrial process conditions.

Zeolite powders are not self-binding; therefore, once formed, they need to be shaped into mechanically stable macroscopic bodies of different sizes and shapes. This is most commonly done by extruding a premixed combination of the zeolite powder and one or more binders. Binders can be organic materials (e.g., organic polymers) or inorganic materials such as alumina, silica, silicates or clays, or mixtures thereof. Generally, 15-30 wt % of a binder is required to provide sufficient strength and attrition resistance for large-scale extended use.

Robustness and strength are important requirements for such zeolite catalyst bodies. Many industrial processes use such catalyst bodies in large “packed beds” and continuously flow a fluid—whether liquid or gas—through the packed bed to contact the reactants with the catalysts. The fluid may subject the catalyst bodies to movement and attrition, generating dust. A build-up of this fine dusty material may block equipment and is one of the principal factors limiting the useful lifespan of such zeolite bodies.

Unfortunately, the addition of materials acting as binders, such as silica or boehmite, especially at the relatively high levels required for providing adequate robustness, can negatively influence the porosity profile and the chemical behaviour (e.g., the Bronsted acidity) of the zeolite bodies. Additionally, or alternatively, the use of relatively high levels of binders may also result in other negative effects, such as blocking the entrance of the zeolite pores especially at the pore opening of the catalysts (causing poorer mass transfer), effect lower catalytic selectivity and undesired chemical reactions, such as coke formation, and combinations thereof. High levels of binder will reduce the level of the zeolite available for catalysis, for example, within the zeolite bodies. All these, alone or in combination, can reduce the effective lifespan and performance of the zeolite bodies.

Zeolite body or bodies that are mostly or completely comprised of zeolite, for example greater than 85% zeolite, or greater than 95% zeolite or even greater than 98% zeolite by weight of the or each zeolite body, are preferable to a zeolite body or bodies containing significant (i.e. >5%) levels of binders or other inert materials. It is highly preferable for a body of a specific zeolite to mostly or wholly comprise that specific zeolite. Zeolite bodies, when used for catalytic purposes, can be doped with catalytic species, such as metal ion species or nanoparticles. However, typical loadings of these metal catalytic species are very low. Zeolite catalysts will often contain less than about 10% by weight of the or each zeolite body of metal catalytic species by weight of the or each zeolite body, for example less than about 5% or less than about 1% of metal catalytic species by weight of the or each zeolite body. Zeolite catalysts may contain about 0.5% to about 10%, or about 1% to about 5%, of metal catalytic species by weight of the or each zeolite body.

Therefore, the development of useful zeolite bodies and the shaping of zeolite powders or particles into technically relevant forms for industry remains a major interest and challenge, with much extant prior art and on-going development. The ideal zeolite body is one that combines high chemical performance (such as catalytic selectivity and rate of reaction) and high density with high robustness and an extended useful lifespan.

The term “robustness” refers to mechanical robustness. This includes resistance to attrition and dust formation. Empirically, robustness can be assessed by placing 5 g of zeolite bodies in an empty glass jar (volume>200 ml) and shaking intensely as possible by hand for 30 seconds. Less robust particles leave visible traces of dust.

Two measures of density are referred to herein, bulk density and envelope density.

Envelope density relates to the density of an individual zeolite body. The envelope density is the mass of a body divided by the envelope volume. The envelope volume is the volume enclosed by an imaginary “envelope” that is wrapped tightly around the body. The envelope volume includes internal pore volume as well as the volume of the actual material forming the body. A test method for measuring envelope density is disclosed below.

Bulk density refers to the mass of the particulate bodies required to fill a defined volume. As the skilled person will appreciate, the bulk density of a relatively large zeolite body is not usefully measured by having one or two bodies in a small vessel, therefore a large number of zeolite bodies is required to measure the bulk density. Commercial literature for zeolite bodies may often list the bulk density of the bodies. As the skilled person will understand, the envelope density of the individual body(ies) contributes to bulk density, along with a packing factor. However, in general, the greater the envelope density of the zeolite bodies, the greater the resulting bulk density of a plurality of these bodies may be.

Known zeolite catalyst body(ies) produced by current processes require a degree of compromise when attempting to balance these different properties and requirements. Improving robustness requires increasing the amount of binder, which detrimentally affects overall chemical performance, volumetric efficacy and catalytic selectivity. Excessive porosity, especially macroporosity, reduces chemical selectivity and catalyst lifetime, density (both envelope and bulk) without a proportionate increase in surface area and hence volumetric efficacy is also reduced, as is the mechanical robustness. The reduced density means that a larger volume of zeolite bodies is needed for a given process and reaction rate. This in turn will increase costs due to the larger size of equipment needed to accommodate the larger volume of zeolite bodies. Increasing macroporosity will very often typically decrease robustness.

For the purposes of clarity, the International Union of Pure and Applied Chemistry (IUPAC) conventions of pore size are used herein. IUPAC defines micropore as being those pores less than 2 nm. Mesopores are defined as being from 2 nm to 50 nm and macropores are those greater than 50 nm.

Pore size can affect catalytic selectivity and lifespan of the zeolite body. In particular, controlling and limiting the proportion of larger pores (the macropores) can help control which chemical species are able to reach the active catalytic sites. Larger pores are helpful in allowing reactant access to the catalytic sites located within the smaller micropores of the zeolite crystals. However, larger pores, especially macropores, can also allow easy access of materials, including undesirable chemical species, such as unwanted by-products, to the catalytic sites. This increases the chances of unwanted reactions happening. In particular, larger macropores are often linked with the problem of “coking” where carbon deposits build up over time within the zeolite body and limit performance and the effective catalyst lifetime. However, limiting the proportion of larger pores reduces the ease of access of reactants to the catalytic sites. The ideal catalytic body is one that has a controlled hierarchical porosity with a controlled and limited level of larger mesopores—but not macropores—in addition to the microporosity. The mesopores are large enough to allow access of the reactants to the active catalytic sites but can still block access of larger, unwanted by-products (which can lead to over-reaction and coking) without an undesired reduction in density.

All current zeolite bodies are complex compromises between competing and sometimes mutually contradictory requirements. An ideal zeolite catalyst body may be one that combines relatively high robustness, high density, high surface area, high catalytic activity and selective, hierarchical porosity without macroporosity, all in a single zeolite body. Current commercially available zeolite bodies typically have higher levels of macroporosity and this contributes to their limited lifespan.

Fabrication of zeolite bodies with high levels of inorganic binders, such as aluminas, silicas and silicates, have been widely reported but can have some or all of the issues highlighted above relating to reduced performance.

A reported attempt to address the above issues utilises, for example, an acid co-hydrolysis route, as well as high temperature crystallisation to produce self-assembled zeolite monoliths comprising large micron-sized crystals. However, such processes result in zeolite monoliths having high levels of macroporosity with a lower bulk density.

A further attempt to address these issues has been the production of so-called “binder-less” zeolite bodies or monoliths. Typically, in one such aspect, after formation of the body, the inorganic binder material—which needs to be alumina or silica based—is converted in-situ to a zeolite. The intent is to convert the inert inorganic binder material into a more useful material, e.g. a zeolite; however, because of the differing processing conditions, the interstitial zeolite cannot be identical to the bulk zeolite and is often a different species with quite different properties. However, it will be more useful than something totally inert, such as the original binder material. Hence, the term “binder-less” may be somewhat misleading and is better referred to as “non-zeolite-binder”-less.

Such processes are complex but also provide limited control of what happens to the binder during processing, and the nature and form of the zeolite which replaces it. The nature and form of the zeolites formed in-situ within an existing body is, however, inevitably different to that of zeolite crystallites formed during a conventional and controlled sol-gel process. Moreover, zeolite formed in-situ may be less optimally formed than said conventionally formed zeolite crystallites forming the bulk of the zeolite body and the in-situ formation may negatively affect the porosity profile, especially the ability to form a hierarchical porosity profile with a controlled level of larger pores.

Another form of “binder-less” zeolite body are those formed using a binder which is then burned off during a subsequent calcination step. Typically, organic materials such as organic polymers, e.g. starch, are used. These organic materials are sometimes referred to in the art as templates when their sole purpose is to leave pores, however for the purposes of this invention they are referred to as binders. The removal of the binder typically leaves larger pores, such as macropores, as the binder burns away. The density of these organic binders is low compared to the density of the zeolite particles so the inclusion of low wt % levels of organic binder can introduce relatively high levels of porosity. The high temperature of the calcination step sinters the zeolite particles together. Additional control of hierarchical porosity can be introduced by including organic bodies of controlled size—for example, lengths of fibres—into zeolite bodies prior to calcination. The high temperature burns these particles away leaving a pore where they had been.

Another form of “binder-less” zeolite body can be generated by taking a preformed substrate body based on silica or alumina and subsequently reacting it with other reactants to convert it in-situ to a zeolite. Many suitable substrate bodies are commercially available for use as supporting substrates for catalysts. Such bodies need to be highly porous with larger pores (to allow access of the reactants to form the zeolite). Hence the macroporosity of the final zeolite body can be high and bulk and envelope densities low.

However, such “binder-less” templating methods inherently result in lower body densities as the fraction of the volume of the zeolite body occupied by the binder is converted to pore volume within the zeolite body. The resulting porosity typically contains a significant and higher proportion of larger pores in addition to the micropores of the zeolite. Control of porosity is limited with such methods. These macropores can help mass transfer but often also deleteriously i) reduce the chemical selectivity and lifespan of the catalyst as many chemical species can easily access the active catalytic sites and ii) reduce the density of the zeolite bodies. Zeolite bodies provided by conventional processes involving burning off of a binder are usually described as having a hierarchical porosity profile. However, such hierarchical porosity profiles typically include an inefficiently high level of meso and macropores.

For the purposes of clarity, “templating” is also the term often used in the art to describe the use of organic and/or inorganic chemical materials in the synthesis of zeolite particles. These materials may also be referred to as “structural directing agents”, however for the purposes of the invention they will be referred to as templates (e.g. organic templates). The template (structural directing agent) is typically removed by calcination. Removal of the template does not contribute to interstitial macroporosity in the zeolite body.

Nanocrystalline zeolite particles (or crystallites) refer to zeolite particles having a mean particle size of less than about 1000 nm, such as particles having a mean particle size of less than about 900 nm or less than about 800 nm or less than about 600 nm or less than about 400 nm or less than about 200 nm or even less than about 100 nm. Nanocrystalline zeolite particles (or crystallites) may have a mean particle size of greater than about 25 nm, or greater than about 50 nm, or greater than about 100 nm, or greater than about 200 nm, or greater than about 300 nm. Preferably, the nanocrystalline zeolite particles (or crystallites) may have a mean particle size of about 100 nm. It will be appreciated that a zeolite body will comprise a plurality of zeolite particles, including nanocrystalline zeolite particles.

The inventors have discovered that controlling the porosity profile of the zeolite bodies, especially by reducing or practically eliminating macroporosity and limiting, but not eliminating, the mesoporosity, increases the chemical selectivity and useful lifespan of the zeolite body, as well as increasing the density (hence volumetric efficacy) of the zeolite body. Use of zeolite bodies having the inventive porosity profiles and physical parameters typically delivers improved performance in chemical processes. The inventors have discovered that suitable zeolite bodies can be formed from nanocrystalline zeolite, rather than the multi-micron sized zeolite particles typically present in most zeolite powders or suspensions, especially when using a slow drying step during formation of the body. The nanocrystalline zeolite particles forming the zeolite bodies preferably have a mean particle size of less than about 1000 nm, or more preferably less than about 900 nm or more preferably less than about 800 nm or more preferably less than about 600 nm or even more preferably less than about 400 nm or even more preferably less than about 200 nm or most preferably less than about 100 nm. Without wishing to be bound by theory, the tight particle packing that can be achieved with the nanocrystalline zeolite is believed to help increase the robustness of the zeolite body and reduce macroporosity.

The nanocrystalline zeolite particles retain their basic integrity when formed into the larger zeolite bodies. The particle size of the nanocrystalline zeolite particles in the zeolite body can be measured in-situ by use of XRD techniques (see later).

Herein, it is demonstrated that nanocrystalline zeolite can be shaped directly into larger, higher-density, self-supported and mechanically stable zeolite bodies with hierarchical micro and ordered mesoporous structures, without the use of high levels of binders. Low levels of binder (such as less than about 5 wt % by weight of the or each zeolite body), or other additives such as catalytic materials, can optionally be included into the zeolite bodies without departing from the invention.

### SUMMARY OF THE INVENTION

Accordingly, in a first aspect, the present invention provides a mesoporous zeolite body or bodies comprising greater than 85% zeolite by weight of the or each zeolite body, preferably greater than 95% or greater than 98% by weight of the or each zeolite body. The or each zeolite body has a maximum internal diameter of from about 0.1 mm to about 50 mm, or from about 0.25 mm to about 50 mm, or from about 0.5 mm to about 50 mm, or from about 0.2 mm to about 45 mm or from about 0.5 mm to about 40 mm or from about 1 mm to about 30 mm or even from about 2 mm to about 6 mm. The or each zeolite body has an envelope density of greater than about 0.7 g/cm³ and less than about 1.8 g/cm³. Preferably macropores comprise less than about 10%, preferably less than about 5%, more preferably less than about 2%, of the envelope volume of the or each zeolite body.

To measure internal diameter of a body, a line is drawn between an edge and an opposing edge of the body. The maximum internal diameter of the body is the length of the longest line that can be drawn within the dimensions of the body without crossing any external surfaces or edges. Typically, the maximum internal diameter of a body is measured by microscopic examination. Suitable equipment for measuring maximum internal diameters include the STM7 range of industrial microscopes from Olympus, for example the STM7-MF, operated according to the manufacturer's instructions.

Advantageously, the present invention provides a zeolite body having a low level of macroporosity. Preferably macropores make up less than about 10%, preferably less than about 5%, more preferably less than about 2%, of the envelope volume of the or each zeolite body as measured by mercury porosimetry. The inventive body provides a beneficial combination of low to substantially no macroporosity, combined with relatively high mesoporosity and/or microporosity.

Advantageously, the zeolite body of the invention provide a relatively high surface area for a given volume whilst controlling the access of chemical species to active catalytic sites, thus improving chemical selectivity and reducing coke formation. Without being bound by theory, it is understood that such zeolite bodies achieve this by having defined physical properties, including higher envelope density and hierarchical porosity profiles lacking significant macroporosity, and consequentially high catalytic performance, along with relatively high strength and improved catalyst lifetimes. The disclosed bodies typically outperform conventional commercial and hierarchically porous zeolite bodies which will typically have significant levels of macropores.

In embodiments, the zeolite body or bodies may have a maximum internal diameter of from about 0.5 mm to about 45 mm, preferably from about 1 mm to about 10 mm, more preferably from about 2 mm to about 6 mm.

The zeolite forming the or each zeolite body may be nanocrystalline zeolite particles (or crystallites) having a mean particle size of less than about 1000 nm, such as particles having a mean particle size of less than about 900 nm or less than about 800 nm or less than about 600 nm or less than about 400 nm or less than about 200 nm or even less than about 100 nm. The zeolite forming the or each zeolite body is may be nanocrystalline zeolite particles (or crystallites) having a mean particle size of greater than about 25 nm, or greater than about 50 nm, or greater than about 100 nm, or greater than about 200 nm, or greater than about 300 nm. Preferably, the nanocrystalline zeolite particles (or crystallites) may have a mean particle size of about 100 nm.

Accordingly, in a second aspect, the present invention provides a mesoporous zeolite body or bodies comprising greater than 85% zeolite by weight of the or each zeolite body, wherein the or each zeolite body has a maximum internal diameter of 0.25 mm to 50 mm; wherein the or each zeolite body has an envelope density of between 0.7 g/cm³ and 1.8 g/cm³; wherein the zeolite forming the or each zeolite body is nanocrystalline zeolite having a mean particle size of less than 1000 nm. Preferably, wherein macropores comprise less than 10% of the envelope volume of the or each zeolite body.

Such particles may have a mean particle size of less than about 900 nm or less than about 800 nm or less than about 600 nm or less than about 400 nm or less than about 200 nm or even less than about 100 nm. The zeolite forming the or each zeolite body is may be nanocrystalline zeolite particles (or crystallites) having a mean particle size of greater than about 25 nm, or greater than about 50 nm, or greater than about 100 nm, or greater than about 200 nm, or greater than about 300 nm. Preferably, the nanocrystalline zeolite particles (or crystallites) may have a mean particle size of about 100 nm.

For the avoidance of doubt, embodiments related to the first aspect of the invention apply mutatis mutandis to the second aspect of the invention.

Advantageously, zeolite bodies according to the second aspect of the invention may display higher density and improved robustness, along with other benefits described herein.

The following embodiments apply equally and separately to the first aspect and second aspect of the invention and are written once purely in the interests of brevity.

In embodiments, the zeolite body or bodies may have an envelope volume from about 0.1 mm³ to about 500000 mm³, more preferably from about 1 mm³ to about 4000 mm³, even more preferably from 30 mm³ to 900 mm³ and even more preferably from 60 mm³ to 500 mm³, 100 mm³ being an example. Such larger zeolite bodies may be easier to handle.

In embodiments, the zeolite body or bodies may have an envelope density of from about 0.7 g/cm³ to about 1.8 g/cm³, preferably of from about 0.7 g/cm³ to about 1.4 g/cm³, preferably of from about 0.8 g/cm³ to about 1.3 g/cm³ or even more preferably of from about 0.9 g/cm³ to about 1.25 g/cm³. Such densities are significantly higher than most commercially available zeolite bodies.

In embodiments, the zeolite bodies may have a bulk density of greater than about 0.6 g/cm³, preferably from about 0.6 g/cm³ to about 1.4 g/cm³, preferably of from about 0.7 g/cm³ to about 1.3 g/cm³, more preferably of from about 0.8 g/cm³ to about 1.2 g/cm³ or even more preferably of from about 0.85 g/cm³ to about 1.15 g/cm³. Such densities are significantly higher than most commercially available zeolite bodies.

The micropore volume to mesopore volume ratio of the or each zeolite body may be from about 1:1 to about 1:8. Preferably, the micropore volume to mesopore volume ratio of the or each zeolite body is from about 1:1 to about 1:5, more preferably from about 1:2 to about 1:4. Recall that micropores refer to pores having a size of less than 2 nm and mesopores refer to pores having a size from 2 nm to 50 nm, as per the IUPAC definitions.

Typically, the or each zeolite body has a micropore volume of from about 0.1 cm³ g⁻¹ to about 0.4 cm³ g⁻¹, preferably from about 0.15 cm³ g⁻¹ to about 0.3 cm³ g⁻¹, or from about 0.2 cm³ g⁻¹ to about 0.25 cm³ g⁻¹. Preferably, the micropore volume is based on N₂ adsorption isotherm measurements at 77K as described herein. The micropore volume is obtained at a relative pressure of P/P₀=0.1.

Typically, the or each zeolite body has a mesopore volume of from about 0.1 cm³ g⁻¹ to about 0.8 cm³ g⁻¹, preferably from about 0.15 cm³ g⁻¹ to about 0.6 cm³ g⁻¹, most preferably from about 0.2 cm³ g⁻¹ to about 0.4 cm³ g⁻¹. Preferably, the mesopore volume is measured by measurements of the N₂ adsorption isotherm at 77K as described herein. The mesopore volume is the volume adsorbed between relative pressures of P/P₀=0.1 and P/P₀=0.98.

The or each zeolite body has a macropore volume of less than about 10% of the envelope volume of the or each zeolite body as measured by mercury porosimetry. Typically, the or each zeolite body has a macropore volume of less than about 7%, preferably of less than about 5%, more preferably of less than about 3%, even more preferably of less than about 1%, of the envelope volume of the or each zeolite body as measured by mercury porosimetry.

Note that, for technical reasons, mercury porosimetry is not suitable for measurement of microporosity or mesoporosity and nitrogen adsorption is unsuitable for measurement of macroporosity.

Optionally, the or each zeolite body has a Brunauer-Emmet-Teller (BET) area of from about 100 m² g⁻¹ to about 900 m² g⁻¹, or from about 150 m² g⁻¹ to about 800 m² g⁻¹, or from about 200 m² g⁻¹ to about 700 m² g⁻¹, or from about 300 m² g⁻¹ to about 600 m² g⁻¹, or from about 350 m² g⁻¹ to about 450 m² g⁻¹.

The or each zeolite body may be an aluminosilicate zeolite body. Preferably, the aluminosilicate zeolite has the chemical formula NaₙAlₙSi₉₆₋ₙO₁₉₂·16H₂O (0<n<27). Aluminosilicate zeolites having this chemical formula include the widely used catalytic zeolite known commercially as Zeolite Socony Mobil-5, ZSM-5. Alternatively, the or each zeolite body may have the chemical formula M_{2/n}OAl₂O₃·xSiO₂·yH₂O, where the charge-balancing non-framework cation M has valence n, x is 2.0 or more, and y is the moles of water in the voids. The or each zeolite body may have a SiO₂:Al₂O₃ ratio of from about 10:1 to about 1000:1, preferably from about 20:1 to 300:1 and even more preferably from 80:1 to about 100:1.

The or each zeolite body may consist of other zeolite types, for example a MFI-type zeolite such as Titanium Silicate-1 or a pure siliceous (e.g. Silicalite-1) zeolite. For example, the or each zeolite forming the zeolite body may be a Faujasite such as Zeolite X and Zeolite Y, or a Zeolite Beta (including aluminosilicate Beta and Sn-Beta) or a Chabazite (such as SSZ-13, Cu-SSZ-13, Fe-SSZ-13 and SAPO-34) or Ferrierite or Sodalite or Zeolite L or Mordenite or ZSM-11 or ZSM-22 or ZSM-23 or MCM-22.

The or each zeolite body may further comprise, either as part of the zeolite framework via ion exchange or impregnation with cations, or as metal oxides, one or more heteroatoms selected from the group consisting of: Cu, Ag, Mg, Ca, Sr, Ti, Zr, Hf, Zn, Cd, B, Al, Ga, Sn, Pb, Pt, Pd, Re, Rh, V, P, Zn, Sb, Rb, Li, Cs, Ag, Ba, Cr, Mo, W, Mn, Re, Fe, Co, Ni, Ge and noble metals.

The or each zeolite body may further comprise counterions to the zeolite. Said counterions may be selected from the group consisting of: sodium, lithium, potassium, iron and copper.

The or each zeolite body may be any shape, although preferably the shape is selected from the group consisting of monolithic bodies having a substantially cylindrical or spherical form.

The or each zeolite body may be produced by concentrating a nanocrystalline zeolite colloidal suspension to obtain a wet nanocrystalline zeolite solid by removing solvent, for instance by centrifugation, preferably as discussed in relation to the third aspect. Typically, the wet nanocrystalline zeolite body has a solids content, by reference to the total weight of the wet nanocrystalline zeolite body, of from about 20% to about 60%, preferably from about 30% to about 50%.

Additionally or alternatively, preferably as discussed in relation to the third aspect, the or each zeolite body may be produced by a controlled drying process comprising i) drying the wet nanocrystalline zeolite body at less than about 50° C., preferably at about 15° C. to about 45° C., preferably from about 20° C. to about 35° C., for example about 25° C., for about 6 hours to about 5 days, preferably from about 1 day to about 4 days, more preferably about 2 days to about 3 days.

Beneficially, zeolite bodies according to the first aspect may display improved selectivity in catalysis and gas separation resulting from the ability to tune the hierarchical porosity profile of the zeolite bodies. In addition, zeolite bodies of the first aspect are substantially binder free, which may avoid the problems associated with binder-containing zeolites.

In a third aspect, the present invention provides a method of preparing one or more zeolite bodies as defined in the first or second aspects of the invention.

The method comprising the steps of: a) mixing two or more zeolite precursors to form an organic template-containing synthesis solution; b) heating the synthesis solution to obtain a nanocrystalline zeolite colloidal suspension; c) concentrating the nanocrystalline zeolite colloidal suspension by a process of centrifugation to obtain a solid wet nanocrystalline zeolite body (in a form of a concentrated gel); d) drying the wet nanocrystalline zeolite body, preferably at less than about 50° C., to form one or more of said zeolite bodies; and e) removing the organic template and obtaining one or more substantially organic template-free zeolite bodies. Advantageously, the centrifugation step may contribute to the uncharacteristically high densities of the zeolite bodies of the invention, improving packing of the nanocrystalline zeolite particles.

Preferably the drying step d) occurs at a relatively low temperature (e.g., less than about 50° C., preferably less than about 45° C.), and is preferably extended over a relatively long period of time (e.g., about a day or more). Without being bound by theory, it is believed that a relatively slow drying process further contributes to the uncharacteristically high densities of zeolite bodies according to the invention by further facilitating the close packing of the nanocrystalline zeolite particles driven by surface tension effects. Fast drying processes, such as those at temperatures greater than 50° C. and/or under vacuum, are typically observed to form powders instead of coherent, dense bodies. Without wishing to be bound by theory, it is believed that the greater stresses from the surface tension of the liquid meniscus moving rapidly through the body as it is rapidly dried disrupts larger-scale structures and results in the formation of fine powder. However, once the free liquid has been dried under low temperatures and the structure formed, higher temperatures can be used to complete the drying since this will not damage the structure. Preferably, step d) is not carried out under vacuum.

Preferably, one of the two or more zeolite precursors is an organic template-containing zeolite precursor. Such template-containing precursors help form the internal zeolite pores. Preferably, the organic template-containing zeolite precursor includes tetraethyl orthosilicate hydrolysed with tetrapropylammonium hydroxide and water.

Typically, in step b) the synthesis solution is heated to a temperature of from about 35° C. to about 100° C., preferably from about 45° C. to about 80° C., more preferably from about 50° C. to about 70° C. for sufficient time to obtain a nanocrystalline zeolite colloidal suspension. Typically, the sufficient time is from about 2 days to about 7 days, preferably from about 4 hours to about 6 days, more preferably from about 2 days to about 4 days.

For example, step b) is a controlled heating process comprising i) heating the solution to a temperature of from about 45° C. to about 100° C., preferably from about 50° C. to about 100° C., or from about 55° C. to about 85° C., or from about 65° C. to about 75° C., for from about 12 hours to about 7 days, preferably from about 1 day to 5 days, and/or ii) subsequently heating the solution to a temperature of from about 35° C. to about 70° C., preferably from about 45° C. to about 70° C., or from about 45° C. to about 60° C., for a further from about 2 to about 7 days, preferably from about 3 to about 5 days.

Preferably, the nanocrystalline zeolite colloidal suspension is an aqueous colloidal zeolite suspension. Typically, the nanocrystalline zeolite colloidal suspension has a solids content, by reference to the total weight of the suspension, of from about 5% to about 30%, preferably from about 10% to about 20%.

In all aspects, the nanocrystalline zeolite may have a mean particle size of less than about 1000 nm, preferably less than about 900 nm, preferably less than about 800 nm, more preferably less than about 600 nm, even more preferably less than about 400 nm, more preferably less than about 200 nm and most preferably less than about 100 nm). In all aspects, the nanocrystalline zeolite may have a mean particle size of greater than about 25 nm, or greater than about 50 nm, or greater than about 100 nm, or greater than about 200 nm, or greater than about 300 nm. Preferably, the nanocrystalline zeolite has a mean particle size of about 100 nm. Typically, the nanocrystalline zeolite suspension may be in form of a gel which is a non-fluid colloidal network or polymer network that is expanded throughout its whole volume by a fluid. A wet nanocrystalline zeolite body is a solid zeolite body containing nanocrystalline zeolite particles that are in intimate contact with each other but still containing liquid both internally and interstitially.

In step c) the nanocrystalline zeolite colloidal suspension is concentrated to obtain a wet nanocrystalline zeolite body by removing solvent and concentrating solid by high-speed centrifugation, typically using forces of between about 2000 g and about 18000 g. Typically, the wet nanocrystalline zeolite body has a solids content, by reference to the total weight of the wet nanocrystalline zeolite body, of greater than about 30% to about 70%, preferably from about 40% to about 60%.

Typically, step d) is a drying process, typically a controlled drying process comprising drying the wet nanocrystalline zeolite body at about 15° C. to about 50° C., preferably from about 20° C. to about 45° C., or from about 25° C. to about 35° C., for example about 25° C., for about 12 hours to about 5 days, preferably about 2 to about 4 days, to form a dried nanocrystalline body.

The or each dried nanocrystalline zeolite body may then be subsequently heated to from about 40° C. to about 60° C., preferably from about 45° C. to about 55° C., for from about 12 hours to about 5 days, preferably from about 1 days to about 3 days.

Preferably, once dried, the one or more zeolite bodies have a water content, by reference to the total weight of the zeolite body, of about 0.1% to about 10%, preferably from about 2% to about 6%. Typically, the water is incorporated into the structure of the zeolite rather than being merely adsorbed on the surface.

Following step d), optionally the one or more zeolite bodies may be calcined to remove any internal organic template and obtain one or more substantially template-free zeolite bodies. “Substantially template-free” means the one or more zeolite bodies may comprise by weight of the zeolite body of less than about 1% template, or less than about 0.1% template, or less than about 0.01% template.

Typically, the one or more zeolite bodies are heated, typically in a muffle furnace, at from about 300° C. to about 700° C., preferably from about 400° C. to about 600° C., for about 2 hours to 7 hours, preferably from about 3 hours to 6.5 hours, for example at about 550° C. for about 6 hours.

Subsequently, the method of the third aspect may include the further step, following calcining, of transforming the one or more calcined zeolite bodies, preferably to its ammonium form, by an ion exchange method. The ion exchange method may comprise the step of mixing the one or more zeolite bodies with an ammonium source, for example aqueous ammonium chloride aqueous solution. Typically, ion exchange method is carried out at from about 25° C. to about 90° C., preferably from about 40° C. to about 90° C., or from about 60° C. to about about 85° C., or from about 75° C. to about 85° C. for about 4 hours to about 24 hours, preferably about 8 hours to about 14 hours.

Optionally, the one or more zeolite bodies may be subsequently dried and heated to remove ammonium ions. Typically, the drying step is carried out at from about 20° C. to about 60° C., preferably from about 30° C. to about 50° C., for about 6 hours to about 5 days, preferably from about 12 hours to about 2 days.

Advantageously, the method of the third aspect may allow the preparation of zeolite bodies without the need for the relatively high levels of binders as required in known zeolite preparation methods and/or complex post-processing steps. In addition, the method of the third aspect may provide zeolite bodies having a higher density and/or porosity than those zeolite bodies produced by known methods.

In a fourth aspect, the present invention provides a method of preparing one or more zeolite bodies having an envelope density of greater than about 0.7 g/cm³, preferably greater than about 0.8 g/cm³. In embodiments, the zeolite body or bodies may have an envelope density of from about 0.7 g/cm³ to about 1.4 g/cm³, preferably of from about 0.8 g/cm³ to about 1.3 g/cm³ or even more preferably of from about 0.9 g/cm³ to about 1.25 g/cm³. Such densities are significantly higher than most commercially available zeolite bodies.

The method of the fourth aspect comprises the steps of: a) mixing two or more zeolite precursors to form an organic template-containing synthesis solution; b) heating the synthesis solution to a sufficient temperature, such as from about 45° C. to about 80° C., for sufficient time to obtain a nanocrystalline zeolite colloidal suspension; c) concentrating the nanocrystalline zeolite colloidal suspension to obtain a wet nanocrystalline (gel) zeolite body; d) drying the wet nanocrystalline zeolite body, preferably at less than about 50° C., to form one or more solid organic template-containing zeolite bodies, preferably having a maximum internal diameter of from about 0.1 mm to about 50 mm or from about 0.25 mm to about 50 mm, or from about 0.5 mm to about 50 mm, and e) heating the one or more organic template-containing zeolite bodies to remove the organic template and obtain one or more substantially template-free zeolite bodies.

Preferably, one of the two or more zeolite precursors is an organic template-containing zeolite precursor including tetraethyl orthosilicate hydrolysed with tetrapropylammonium hydroxide and water.

Typically, in step b) the synthesis solution is heated at a temperature of from about 35° C. to about 100° C., preferably from about 45° C. to about 80° C., or from about 60° C. to about 70° C. for sufficient time to obtain a nanocrystalline zeolite colloidal suspension. Typically, the sufficient time is from about 12 hours to about 7 days, preferably from about 1 days to about 6 days, more preferably from about 2 days to about 4 days.

For example, step b) is a controlled heating process comprising i) heating the solution to a temperature of from about 45° C. to about 100° C., preferably from about 50° C. to about 100° C., or from about 55° C. to about 85° C., or from about 65° C. to about 75° C., for from about 12 hours to about 7 days, preferably from about 24 hours to about 5 days, and/or ii) subsequently heating the solution to a temperature of from about 35° C. to about 80° C., from about 45° C. to about 70° C., or preferably from about 55° C. to about 65° C., for a further from about 12 hours to about 7 days, preferably from about 2 to about 5 days.

Preferably, the nanocrystalline zeolite colloidal suspension is an aqueous colloidal zeolite suspension. Typically, the nanocrystalline zeolite colloidal suspension has a solids content, by reference to the total weight of the suspension, of from about 5% to about 30%, preferably from about 10% to about 20%.

Typically, in step c) the nanocrystalline zeolite colloidal suspension is concentrated to obtain a wet nanocrystalline zeolite solid by removing solvent, for instance by centrifugation, preferably as discussed in relation to the third aspect. Typically, the wet nanocrystalline zeolite body has a solids content, by reference to the total weight of the wet nanocrystalline zeolite body, of from about 20% to about 60%, preferably from about 30% to about 50%.

Typically, step d) is a controlled drying process comprising i) drying the wet nanocrystalline zeolite body at less than about 50° C., preferably at about 15° C. to about 45° C., preferably from about 20° C. to about 35° C., for example about 25° C., for about 6 hours to about 5 days, preferably from about 1 day to about 4 days, more preferably about 2 days to about 3 days.

The or each dried nanocrystalline zeolite body may then be subsequently heated to from about 25° C. to about 60° C., preferably from about 45° C. to about 60° C., more preferably from about 35° C. to about 55° C., for from about 6 hours to about 5 days, preferably from about 12 hours to about 4 days, more preferably from about 1 to about 3 days.

The obtained one or more solid organic template-containing zeolite bodies preferably have a maximum internal diameter of from about 0.1 mm to about 50 mm, preferably from about 0.5 mm to about 50 mm, more preferably from about 0.5 mm to about 40 mm and even more preferably from about 1 mm to about 10 mm. Preferably, the one or more solid organic template-containing zeolite bodies have a water content of about 1% to about 10%, preferably from about 2% to about 8%.

Typically, step e) removes organic template from the one or more organic template-containing zeolite bodies to obtain one or more substantially template-free zeolite bodies. “Substantially template-free” means the one or more zeolite bodies may comprise by weight of the zeolite body of less than about 1% template, or less than about 0.1% template, or less than about 0.01% template.

Typically, the one or more organic template-containing zeolite bodies are calcined in a muffle furnace at from about 300° C. to about 700° C., preferably from about 400° C. to about 650° C., for about 4 hours to about 9 hours, preferably from about 5 hours to about 8 hours. For example, at about 550° C. for about 6 hours.

The method of the fourth aspect may further comprise the step of f) transforming the calcined zeolite body, preferably to its ammonium form, by an ion exchange method, preferably followed by drying and optionally further calcining the zeolite body. Secondary calcination produces protonated zeolite.

The ion exchange method may comprise the step of mixing the one or more zeolite bodies with an ammonium source, for example aqueous ammonium chloride solution. Typically, ion exchange method is carried out at from about 25° C. to about 90° C., preferably from about 40° C. to about 90° C., or from about 60° C. to about 85° C., or from about 65° C. to about 85° C. for about 6 hours to about 24 hours, preferably about 12 hours to 18 hours. Typically, the ion exchange method is carried out twice, with the one or more zeolite bodies optionally being washed with water between each ion exchange method. Typically, the pH is then adjusted to from about pH 7 to about pH 8.

Typically, the drying step following step f) is carried out at from about 20° C. to about 60° C., preferably from about 30° C. to about 55° C., for about 12 hours to about 5 days, preferably from about 1 day to about 3 days. Typically, the calcining step following step f) is carried out in a muffle furnace at from about 300° C. to about 700° C., preferably from about 400° C. to about 650° C., for about 4 hours to about 9 hours, preferably from about 5 hours to about 8 hours. For example, at about 550° C. for about 6 hours.

Advantageously, the method of the fourth aspect may allow the preparation of zeolite bodies without the need for high pressure or binders as required in known zeolite preparation methods. In addition, the method of the fourth aspect may provide zeolite bodies having a higher density and lower porosity than those zeolite bodies produced by known methods.

In a fifth aspect, the present invention provides a zeolite body or bodies manufactured according to the methods of the third or fourth aspects.

In a sixth aspect, the present invention provides the use of a zeolite body or bodies according to the first or second aspect, or a zeolite body or bodies prepared according to the methods of the third or fourth aspects in catalysis or adsorption.

In a seventh aspect, the present invention provides a plurality of zeolite bodies according to the first, second or fifth aspects, or manufactured according to the methods of the third or fourth aspects wherein the zeolite bodies have a bulk density of greater than 0.6 g/cm³.

For the avoidance of doubt, embodiments of each aspect of the invention apply mutatis mutandis to other aspects of the invention.

### BRIEF DESCRIPTION OF THE DRAWINGS

The invention will now be described with reference to the following figures which are intended to be non-limiting.

FIG. 1 Digital photographs of H-ZSM-5 bodies with different particle sizes: a. cm-sized, b. 1-2 mm-sized and c. 0.5-1 mm-sized H-ZSM-5 bodies.

FIG. 2 PXRD pattern of calcined H-ZSM-5 body sample. The inset shows the MFI framework structure.

FIG. 3 Gas adsorption characterisation. a. Nitrogen physisorption isotherm of H-ZSM-5 body sample, b. BJH pore size distribution curve obtained from the adsorption branch, and c. Classical DFT pore size distribution curve.

FIG. 4 Conversion of methanol and product selectivity versus time-on-stream: a. H-ZSM-5 crushed body and b. 1-2 mm sized H-ZSM-5 body. Weight Hour Space Velocity (WHSV)=8 h⁻¹, T=450° C.

### DETAILED DESCRIPTION

Throughout this specification, one or more aspects of the invention may be combined with one or more features described in the specification to define distinct embodiments of the invention.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps. The term “comprising” includes within its ambit the term “consisting” or “consisting essentially of”.

The term “consisting” or variants thereof is to be understood to imply the inclusion of a stated element, integer or step, or group of elements, integers or steps, and the exclusion of any other element, integer or step or group of elements, integers or steps.

Unless stated otherwise, the term “consisting essentially of” or variants thereof is to be understood to imply the inclusion of a stated element, integer or step, or group of elements, integers or steps, and that further components may be present, but only those not materially affecting the essential characteristics of the or each zeolite bodies, methods, or uses.

The present invention relates to zeolite bodies, methods of manufacturing said zeolite bodies, and uses of said zeolite bodies, in particular in catalysis and gas separation.

Zeolites are three-dimensional, microporous crystalline materials with well-defined structures of voids and channels of discrete size, which is accessible through pores of well-defined molecular dimensions. Aluminosilicate zeolites contain aluminium, silicon, and oxygen in their regular framework. Titanium silicate zeolites contain titanium, silicon and oxygen in their regular framework.

When referring to zeolite bodies, “body”/“bodies” may be used interchangeably with “monolith”/“monoliths”, and the like. [101]“Nanocrystalline zeolite” refers to zeolite having a mean particle size smaller than about 1000 nm.

A “nanocrystalline zeolite gel” is a non-fluid colloidal network that is expanded throughout its whole volume by a fluid.

Preferred zeolite bodies according to the present invention may be aluminosilicate zeolites having the chemical formula NaₙAlₙSi₉₆₋ₙO₁₉₂·16H₂O (0<n<27) or the chemical formula M_{2/n}OAl₂O₃·xSiO₂·yH₂O, where the charge-balancing non-framework cation M has valence n, x is 2.0 or more, and y is the moles of water in the voids. A particularly preferred commercial zeolite having the first general formula is known as ZSM-5 zeolite.

Envelope density may be measured according to the method disclosed herein. The envelope density of the one or more zeolite bodies may be from about 0.7 g/cm³ to about 1.4 g/cm³, more preferably from about 0.7 g/cm³ to about 1.2 g/cm³, even more preferably from about 0.8 g/cm³ to about 1.0 g/cm³.

Hierarchical porous materials, such as the zeolite bodies disclosed herein, exhibit at least two types of pore systems that have sizes in distinctly different ranges, e.g. in the micropore range and the mesopore range. Microporosity/micropore/microporous relate to pores in a material having a diameter of less than 2 nm. Mesoporosity/mesopore/mesoporous relate to the pores of a material having a diameter of from 2 nm to 50 nm. Macroporosity/macropore/macroporous relate to the pores of a material of greater than 50 nm in diameter.

Mesoporous zeolite body(ies) relates to a zeolite body(ies) having a hierarchical porosity profile comprising pores in the micropore range and the mesopore range.

As used herein “calcining” and/or “calcination” relates to heating a solid to high temperatures, preferably in absence of air or oxygen, generally for the purpose of removing impurities or volatile substances.

Preferably the zeolite bodies are substantially binder free. “Substantially binder free” means the zeolite bodies may comprise by weight of the zeolite body of less than about 5% binder material or less than about 3% binder material, or less than about 0.1% binder material. Typical zeolite binders may be alumina, silica or clays. For examples of known binders see, for instance, Bingre, R. et al., Catalysts 2018, 8, 163; Seghers, S. et al. ChemSusChem, 2018, 11, 1686-693; Lefevere, J. et al. Chem. Pap. 2014, 68, 1143-1153; and Zečević, J. et al. Nature 2015, 528, 245-248. Binder(s) may include the in-situ converted zeolite binder(s) described hereinbefore, preferably the zeolite body is substantially free of such binders.

Additionally, or alternatively, “substantially template-free” means the one or more zeolite bodies may comprise by weight of the zeolite body of less than about 1% template, or less than about 0.1% template, or less than about 0.01% template.

Preferably, the or each zeolite body according to the invention consist essentially of fused nanocrystalline zeolite, preferably the or each zeolite body according the invention consists of fused nanocrystalline zeolite. That is to say, adjacent nanocrystalline particles within the zeolite body are bonded directly to each other.

Preferably, the or each zeolite body of the present invention consist essentially of zeolite, preferably the or each zeolite body consists of greater than about 85% zeolite by weight of the or each zeolite body, preferably 95% zeolite by weight of the or each zeolite body, more preferably greater than about 98% weight of the or each zeolite body, most preferably greater than about 99% weight of the or each zeolite body.

Additionally, or alternatively, the or each zeolite body of the present invention consist essentially of a single phase zeolite, preferably the or each zeolite body consists of a single phase zeolite. Preferably, the or each zeolite body contains by weight of the or each zeolite body of less than about 5% by weight of the body of secondary zeolite and/or non-zeolite, preferably less than about 3% by weight of the body of secondary zeolite and/or non-zeolite, more preferably less than about 1% by weight of the body of secondary zeolite and/or non-zeolite. A secondary zeolite is understood to be a zeolite other than that forming the bulk structure of the nanocrystalline zeolite(s) from which the or each zeolite body is formed (i.e. the primary single phase zeolite).

The zeolite bodies of the present invention may have applications in catalysis, in particular in gas separation, gas adsorption or the MTO reaction.

### Methanol-to-Olefins (MTO) Reaction

The methanol-to-olefins (MTO) reaction has long been considered as a preferable alternative route to produce valuable light olefins (e.g. ethylene and propylene) over traditional thermal cracking of naphtha because methanol can be easily produced from non-oil resources, such as coal and natural gas. However, methanol is very sensitive to acidic zeolite catalysts due to their high activity, which could further catalyse the direct C—C bonds into a large variety of hydrocarbon by-products inside the zeolite pores. This makes the reaction complicated and difficult to control over different zeolite framework types. Having a zeolite body that substantially or wholly comprises a single type of zeolite may aid in reaction control.

The MTO reaction is commonly catalysed over microporous zeolites, such as ZSM-5 and SAPO-34. SAPO-34, which possesses a CHA framework with small channels and cages, is highly selective toward light olefins production (>90%) at 100% methanol conversion. However, a drawback associated with SAPO-34 is a rapid deactivation due to coke deposition. Medium pore size aluminosilicate ZSM-5 zeolite catalyst with ten-membered ring pores has a longer catalyst lifetime, however lower yield of light olefins (<50%) are produced as compared to SAPO-34. A long-standing challenge in MTO process has been to design a zeolite catalyst that could transform methanol with high light olefins selectivity at 100% methanol conversion with a long catalyst lifetime.

Since the first discovery of MTO reaction over aluminosilicate ZSM-5 zeolite catalysts by Mobil's researchers in 1977, large-scale industrialisation of the MTO process has been implemented in the past decade. Olefin selectivity and resistance to coking are the two most important parameters in optimising the catalyst performance for this typical process. Various strategies have been employed to increase the selectivity towards light olefins and simultaneously improve catalyst lifetime of ZSM-5 zeolite by optimising the acidity, scaling down the crystal size, introducing secondary mesopore system, and doping the pore structure with heteroatoms. The effect of acidity on the performance of the ZSM-5 for the production of propylene has recently been investigated. It was demonstrated that the isolation of Bronsted acid sites via acid leaching is a key parameter to the selective formation of propylene while the introduction of Lewis acid sites via the incorporation of calcium or magnesium prevents the formation of coke, hence drastically increasing catalyst lifetime.

In addition, the shaping of ZSM-5 zeolite with different binders (e.g. silica or boehmite) may cause significant alteration of the zeolite Brønsted acid sites, resulting in higher amounts of coke formation, as well as reduction in the catalyst lifetime and light olefins selectivity in MTO reactions.

The zeolite bodies of the present invention demonstrate improved light olefin selectivity and improved catalyst lifetime in the MTO reaction compared to the known zeolites.

### Examples

The invention will now be demonstrated by reference to the following non-limiting examples.

Unless otherwise mentioned, room temperature and pressure are 20° C. (293.15 K, 68° F.) and 1 atm (14.696 psi, 101.325 kPa), respectively. For the purposes of the invention, measurements are made in these conditions unless otherwise mentioned.

### MTO Reaction

An MTO reaction was carried out at 450° C. with Weight Hour Space Volume (WHSV)=8 h⁻¹ over crushed and 1-2 mm sized H-ZSM-5 bodies manufactured according to the method set out below. The catalyst lifetime is defined as the time for which the conversion of methanol exceeded 90%. FIG. 4 shows the full methanol conversion over crushed and 1-2 mm sized H-ZSM-5 body. However, 1-2 mm sized H-ZSM-5 body exhibits a longer catalyst lifetime compared to the crushed H-ZSM-5 body. The time taken for the methanol conversion to drop below 95% conversion was 105 h for the 1-2 mm sized H-ZSM-5 body versus 90 h observed for the crushed H-ZSM-5 body. H-ZSM-5 body achieves a higher light olefin selectivity (C₂-C₄=selectivity=70%) as compared to its crushed form (C₂-C₄=selectivity=65%). The H-ZSM-5 body shows superior catalytic performance in comparison to its crushed from with an improved light olefin (C₂-C₄═) selectivity and improved catalyst lifetime.

Known zeolites powders or known zeolite bodies, optionally produced by an extrusion approach, do not achieve such high light olefin selectivity in the MTO reaction. Additionally, these known zeolites (powders or bodies) display shorter catalyst lifetimes.

### Experimental Methods

### Method for Measuring the Envelope Density of a Body

The envelope density of a body can be measured by dividing the weight of a body (in grams) by its envelope volume (in mm³). The envelope volume is defined in ASTM D3766 as “the ratio of the mass of a particle to the sum of the volumes of the solid in each piece and the voids within each piece, that is, within close-fitting imaginary envelopes completely surrounding each piece”. The envelope density of a body can be measured using techniques based on the Archimedes principle of volume displacement. The envelope density can be measured by mercury porosimetry. At atmospheric pressure, mercury does not intrude into internal pores. Therefore, the volume of mercury displaced by a body at atmospheric pressure is the envelope volume of the body. Dividing the weight of the sample by this volume gives the envelope density. The use of mercury porosimetry is described below.

An alternative technique for larger bodies, those with a diameter>5 mm, is to use accurate 3-D scanners to measure the body volume. Suitable equipment includes the Leica BLK360.

### Method of Measuring the Bulk Density of a Plurality of Bodies

The bulk density of a plurality of bodies can be measured by completely filling a suitable cylindrical vessel of known volume and measuring the mass. The skilled person will appreciate dimensions of the vessel are not fixed as an appropriate size of vessel will be dependent on the dimensions of the bodies being measured, both the internal diameter and height of the vessel should each be greater than ten times the maximum internal dimension of the bodies. There is no restriction on upper limit on the volume of the vessel, although larger volumes may require prohibitively expensive amounts of material to fill them.

The mass and volume of the empty vessel are measured. The vessel is then filled by pouring in the zeolite bodies into the vessel from 5 cm above the height of the vessel. The top of the poured zeolite bodies is levelled with the top of the vessel. The combined mass of the vessel and the contained zeolite bodies is then measured. The mass of the zeolite bodies is calculated by deducting the mass of the empty vessel from the combined mass of the vessel and zeolite bodies. The bulk density is then calculated by dividing the mass of the zeolite bodies by the volume of the vessel.

### Method of Measuring Nanocrystalline Zeolite Mean Particle Size

The average particle size of the nanocrystalline zeolites (nanocrystalline zeolite particles) in the zeolite body can be determined by X-ray diffraction using the Scherrer equation to calculate crystallite size from the full-width at half maximum (FWHM) measure of the diffraction peak. Zeolite crystallites are often quite isotropic in shape with no single preferred orientation of crystallite growth so the choice of which reflection to use is not critical but, for consistency, the (0 1 0) reflection is used, and the K value is constant at 0.94. Suitable equipment includes the X'Pert Pro from PANalytical, which is used according to the manufacturer's guidelines.

The average particle size of the nanocrystalline zeolite particles in the zeolite colloidal suspension formed in step b) can be measured by Dynamic Light Scattering. Suitable equipment includes the NANO-flex II from Colloid Metrix. Dilution of the suspension is not required.

### Method for Measuring the BET Surface Area of the Body.

The BET surface area of a monolith body can be measured by use of ASTM method D3663-03 “Standard test method for surface area of catalysts and catalyst carriers”. The BET surface area is determined by measuring the volume of nitrogen gas adsorbed at various low-pressure levels by the monolith sample. Pressure differentials caused by introducing the monolith surface area to a fixed volume of nitrogen in the test apparatus are measured and used to calculate BET surface area. Suitable equipment for measuring BET surface areas include the 3Flex from Micromeritics Corporation, used according to the manufacturer's guidelines.

### Method of Measuring Micropore and Mesopore Volume

The micro and meso-porosity profile of a body can be determined by test method ASTM D4641-17. Suitable equipment for carrying out such tests is the ASAP 2020 Plus, from Micromeritics Corporation. The test method is as follows.

The test sample (0.5 g) is typically heated to 300° C. under vacuum to remove adsorbed gases and vapours from the surface. The nitrogen adsorption branch of the isotherm is then determined by placing the sample under vacuum, cooling the sample to the boiling point of liquid nitrogen (˜77.3 K), and then adding, in a stepwise manner, known amounts of nitrogen gas at increasing pressure P to the sample in such amounts that the form of the adsorption isotherm is adequately defined and the saturation pressure of nitrogen is reached.

Each additional dose of nitrogen is introduced to the sample only after the preceding dose of nitrogen has reached adsorption equilibrium with the sample.

By definition, equilibrium is reached if the change in gas pressure is no greater than 0.1 torr/5 min interval. This is continued until P₀ (the gas saturation pressure) is reached.

Data is typically plotted as the amount of gas adsorbed/desorbed (and derived porosity profiles) as a function of P/P₀. The desorption isotherm is determined by desorbing nitrogen from the saturated sample in a stepwise manner with the same precautions taken to ensure desorption equilibration as those applied under adsorption conditions. Microporosity is associated with the volume of gas adsorbed at P/P₀ values of <0.1 whereas mesoporosity is associated with the volume of gas adsorbed at P/P₀ values between 0.1 and 0.98.

### Method of Measuring Macroporosity by Mercury Porosimetry

Mercury porosity values can be measured according to ASTM D4284-12. Suitable equipment for carrying out ASTM D4284-12 include the Micromeritics AutoPore VI 9510 from Micromeritics Corp, USA. The surface tension and contact angle of mercury are taken as being 485 mN/m and 130°, respectively. In ASTM D4284-12, mercury is forced into pores under pressure. A sample size of 1 g is used.

The pressure required to force mercury into the pores of the sample is inversely proportional to the size of the pores according to the Washburn equation. It is assumed that all pores are cylindrical for the purpose of characterization. The porosimeter increases the pressure on the mercury inside the sample holder to cause mercury to intrude into increasingly small sample pores. The AutoPore VI will automatically translate the applied pressures into equivalent pore diameters using the Washburn equation and the values of contact angle and surface tension given above.

The envelope volume of the sample is determined by the volume of mercury displaced at atmospheric pressure. As the applied pressure is increased, mercury is forced into internal pores. The % macro-porosity of a sample is therefore the volume of the mercury intruded into the sample as the pressure is increased from 1.001 atm to 292 atm as a proportion of the volume displaced at atmospheric pressure.

### Methanol-to-Olefin Reaction Method

Catalytic experiments were carried out in a Microactivity Reference unit (PID Eng&Tech). The zeolite catalyst (1-2 mm) was mixed with SiC (6:1 wt. %) and placed in a fixed-bed with an internal diameter of 9 mm for standard experiments. An ISCO pump was used to feed methanol to the reactor system. A weight-hourly space velocity (WHSV) of 8 g MeOH gcat⁻¹ h⁻¹, an N₂:MeOH=1:1 molar feed composition and atmospheric pressure were utilised. The product mixture was analysed online with an Interscience CompactGC equipped with a 15 m capillary RTX-1 (1% diphenyl-, 99% dimethylpolysiloxane) column and a flame ionisation detector.

### MATERIALS AND SYNTHESIS EXAMPLES

All reagents unless otherwise stated were obtained from commercial sources and were used without further purification.

### Materials

Tetraethyl orthosilicate, TEOS (reagent grade, 98%), Sigma Aldrich; Tetrapropylammonium hydroxide solution, TPAOH 1.0 M in H₂O, Sigma Aldrich; NaOH pellets (98%), Alfa Aesar; Aluminium sulfate-18-hydrate, Al₂(SO₄)·18H₂O (>94%), Fisher Scientific; Ammonia solution 35%, Fisher Scientific; Ammonium chloride 99.6%, Acros Organic.

### Example 1—ZSM-5 Monoliths

A TPA-silicate solution containing 10 g of TEOS hydrolyzed in 12 g of TPAOH was prepared under vigorous magnetic stirring at room temperature overnight. Another solution containing 5 g of TPAOH, 0.07 g NaOH and 2 g of distilled water was prepared.

TPA-aluminate was then prepared by adding freshly prepared Al(OH)₃ gel according to method reported in Schoeman, B. J., et al., Zeolites 1994, 14, 110-116. The Al(OH)₃ gel was prepared via precipitation from an aqueous Al₂(SO₄)₃ solution with ammonia. The gel was obtained and washed by repeated centrifugation at a speed of 12000 rpm (equivalent to 2500 g) and re-dispersion in distilled water until pH 7.

The gel was weighed once again to determine the weight of the Al(OH)₃ filter cake. It was thereby possible to calculate the water content in the filter cake assuming that it consisted of Al₂(SO₄)₃ and water. The water content was calculated to be ca. 40 wt. %. The TPA-aluminate solution was added dropwise to the silica solution with vigorous stirring to obtain a homogeneous synthesis solution. The solution was heated in an oven at 70° C. in a Schott Duran bottle for 5 days and transferred to a pre-heated oven at 50° C. for another 5 days. After the synthesis, the ZSM-5 nanocrystals were purified by repeated high-speed centrifugation (12,000 rpm equivalent to 2500 g) and re-dispersion in distilled water until pH 7. The gel was dried at 25° C. for 3 days to obtain the transparent monolithic structure. The monolith was further dried at 50° C. for 3 days and heated to remove the organic template in a muffle furnace at 550° C. for 6 h with a ramp rate of 1° C./min.

The calcined Na-ZSM-5 monolith was then transformed into the ammonium form by a two-fold ion exchange procedure with 0.2 M aqueous ammonium chloride solution under static conditions at 80° C. overnight. The NH₄—ZSM-5 monolith was dried at room temperature for 3 days and followed by drying at 50° C. NH₄—ZSM-5 monolith was calcined at 550° C. in a muffle furnace for 6 h with a ramp rate of 1° C./min to achieve the H-ZSM-5 form.

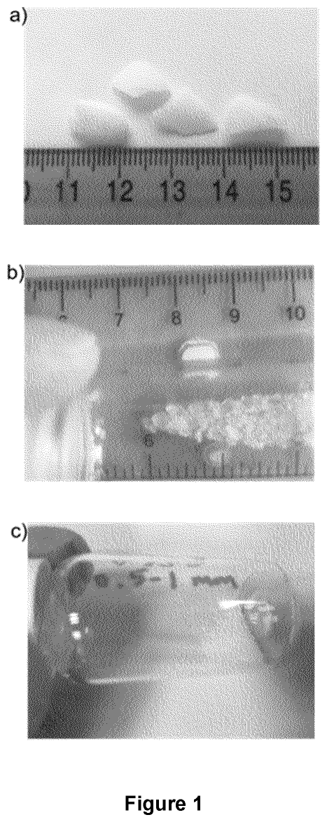

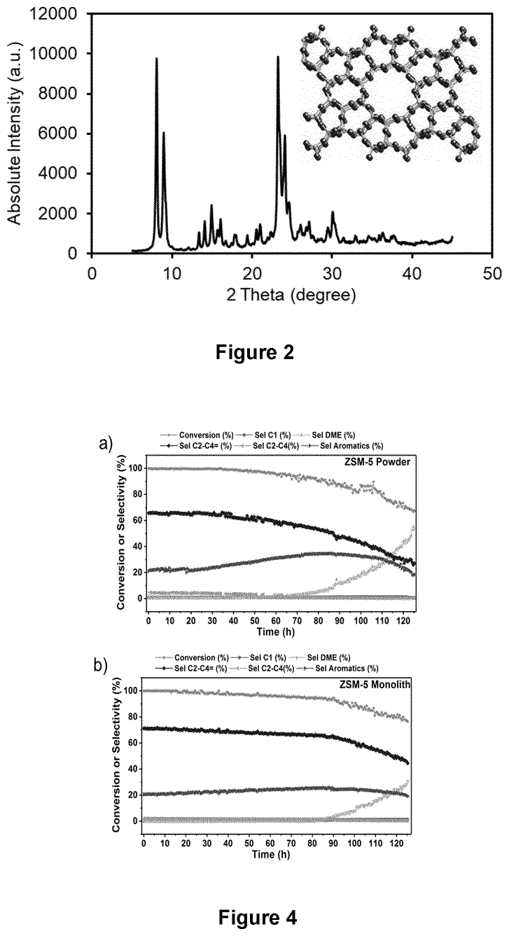

The resulting monolith was then crushed with mortar and pestle and sieved into smaller monolithic structures of 0.5-1 or 1-2 mm size range to obtain two series of H-ZSM-5 zeolites with different particle size ranges. The 1-2 mm structures were used for mercury porosimetry testing. FIG. 1 shows the optical images of the prepared zeolite bodies by way of illustration. The ruler is shown for scale. The original cm-sized bodies were crushed and sieved into mm-sized monoliths with desired particle sizes without the need for binder or extrusion. FIG. 2 shows the PXRD pattern of H-ZSM-5 monolithic sample, confirming its pure phase MFI structure with high crystallinity.

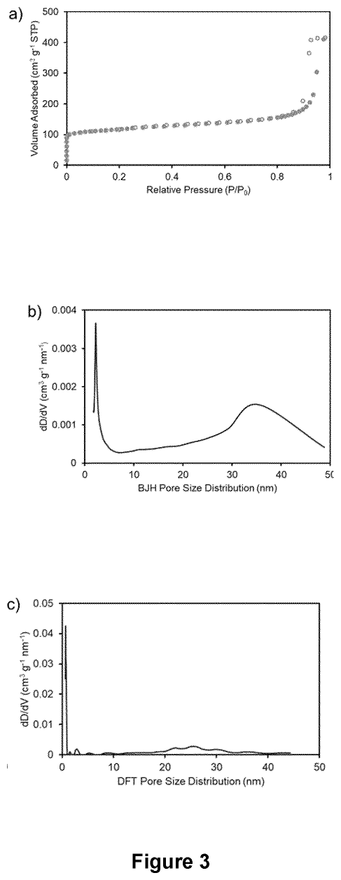

The H-ZSM-5 monolith sample exhibited a combination of Type I and Type IV isotherms (FIG. 3a), with obvious steep uptake in the higher relative pressure range. The observed type H1 hysteresis loop suggests the presence of ordered mesostructures originated from the capillary condensation in the interparticle voids. The corresponding size distribution data calculated from the nitrogen adsorption isotherm by the Barrett-Joyner-Halenda method revealed a bimodal pore-volume distribution with an average mesopore size of 2 nm and 35 nm in diameter.

The H-ZSM-5 monolith sample had a maximum internal diameter of 12 mm and a Brunauer-Emmet-Teller (BET) area, micropore and mesopore volume of 415 m² g⁻¹, 0.16 and 0.46 cm³ g⁻¹, respectively. There was no measurable macroporosity and envelope density was 0.9 g/cm³. This was the first success in the preparation of self-supported zeolite monoliths with ordered mesostructures that are formed via close-packing of nanocrystalline zeolites without applied pressures, binders or replicas.

### Example 2—FAU Zeolite:Zeolite Y Monolith Preparation According to the Published Method in Cryst. Growth Des. 2017, 17, 1173-1179

Solution 1 containing dissolved 5.68 g of NaOH (97+%, ACS reagent, pellet, Acros Organics) and 0.87 g of sodium aluminate (technical, anhydrous, Sigma-Aldrich) in 23.24 g of distilled water was prepared.

Ice-cooled 10.21 g of LUDOX® AS-40 colloidal silica (40 wt. % suspension in H₂O, Sigma-Aldrich) was added dropwise into ice-cooled solution 1 under vigorous magnetic stirring. Solution 1 was then aged statically (i.e. without stirring) at room temperature for 7 days. After 7 days, an additional 14.29 g of LUDOX® AS-40 colloidal silica (40 wt. % suspension in H₂O, Sigma-Aldrich) was added dropwise into solution 1 under vigorous magnetic stirring. The solution was aged statically at room temperature for another 10 days and then thermally treated in a Schott bottle placed in a pre-heated synthesis oven at 60° C. for 16 h.

After the synthesis, the zeolite Y nanocrystals were purified by repeated centrifugation (5250×g) and re-dispersion in distilled water until pH 7-8. The gel was dried at 25° C. for 3 days to obtain the monolithic structure. The monolith was further dried at 50° C. for 3 days and calcined at 550° C. in a muffle furnace for 6 h with a ramp rate of 1° C./min.

Zeolite Na-Y monolith was then transformed into the ammonium form by a two-fold ion exchange procedure with 0.2 M aqueous ammonium chloride solution under static conditions at 80° C. overnight. The NH₄—Y monolith was dried at room temperature for 3 days and then at 50° C. for 3 days. NH₄—Y monolith was calcined at 550° C. in a muffle furnace for 6 h with a ramp rate of 1° C./min to achieve the H-Y form.

Na-Y zeolite monolith has a BET area of 847 m²/g. The calculated micropore volume is 0.25 cm³/g according to the t-plot analysis. The mesopore volume is 1 cm³/g. The NL-DFT pore size distribution curve reveals an average mesopore size of 18 nm.

### Example 3—BEA Zeolite:Zeolite Beta Monolith Preparation

0.21 g of NaOH (97+%, ACS reagent, pellet, Acros Organics) was dissolved in 25.2 g of tetraethylammonium hydroxide (35 wt. % in H₂O, Sigma-Aldrich) to form solution 1. 0.17 g of aluminum isopropoxide (98%, Sigma-Aldrich) was then dissolved in solution 1 under vigorous magnetic stirring. 33.3 g of LUDOX® AS-30 colloidal silica (30 wt. % suspension in H₂O, Sigma-Aldrich) was added dropwise to solution 1 under vigorous stirring for 2 h to achieve a clear solution 1.

Clear synthesis solution 1 was statically aged at room temperature for 48 h and heated at 100° C. in a synthesis oven for 6 days. 1.13 g of aluminum isopropoxide (98%), Sigma-Aldrich was then added to solution 1 and stirred at room temperature for 2 h. The synthesis mixture was heated in a preheated synthesis oven at 100° C. in a Schott bottle for 4 days. After the solvothermal synthesis, the Beta nanocrystals were purified by repeated centrifugation (5250×g) and re-dispersion in distilled water until pH 7-8.

The gel was dried at 25° C. for 3 days to obtain the monolithic structure. The monolith was further dried at 50° C. for 3 days and heated to remove the organic template in a muffle furnace at 550° C. for 6 h with a ramp rate of 1° C./min.

The calcined Na-Beta monolith was then transformed into the ammonium form by a two-fold ion exchange procedure with 0.2 M aqueous ammonium chloride solution under static conditions at 80° C. overnight. The NH₄—Beta monolith was dried at room temperature for 3 days and then at 50° C. for 3 days. NH₄—Beta monolith was calcined at 550° C. in a muffle furnace for 6 h with a ramp rate of 1° C./min to achieve the H-Beta form.

H-Beta zeolite monolith has a BET area of 831 m²/g. The calculated micropore volume is 0.21 cm³/g according to the t-plot analysis. The mesopore volume is 0.45 cm³/g. The NL-DFT pore size distribution curve reveals an average mesopore size of 40 nm.

### Example 4—LTA:Zeolite a Monolith Preparation According to the Reported Method Published in Journal of Sol-Gel Science and Technology, 2021, 98, p. 411-421 with a Slight Modification

0.15 g of NaOH (97+%, ACS reagent, pellet, Acros Organics) was dissolved in 10.4 g of tetramethylammonium hydroxide (25 wt. % in H₂O, Sigma-Aldrich) and 4 g of distilled water under vigorous stirring to form solution 1.

Solution 1 was divided into solution A (8.55 g) and solution B (6 g). 0.85 g of aluminum isopropoxide (98%, Sigma-Aldrich) was dissolved in solution A under vigorous magnetic stirring. 3.83 g of LUDOX® AS-40 colloidal silica (40 wt. % suspension in H₂O, Sigma-Aldrich) was added to solution B to form a clear solution.

Solution A was then added dropwise to solution B under vigorous magnetic stirring. The synthesis mixture was statically aged in a Schott bottle at room temperature for 7 days and thermally treated in a preheated synthesis oven at 100° C. for 24 h. After the solvothermal synthesis, the zeolite A nanocrystals were purified by repeated centrifugation (5250×g) and re-dispersion in distilled water until pH 7-8.

The gel was dried at 25° C. for 3 days to obtain the monolithic structure. The monolith was further dried at 50° C. for 3 days and heated to 550° C. to remove the organic template in a muffle furnace for 6 h with a ramp rate of 1° C./min.

The calcined Na-A monolith was then transformed into the ammonium form by a two-fold ion exchange procedure with 0.2 M aqueous ammonium chloride solution under static conditions at 80° C. overnight. The NH₄-A monolith was dried at room temperature for 3 days and then at 50° C. for 3 days. NH₄-A monolith was calcined at 550° C. in a muffle furnace for 6 h with a ramp rate of 1° C./min to achieve the H-A form.

Na-A zeolite monolith has a BET area of 538 m²/g. The calculated micropore volume is 0.20 cm³/g according to the t-plot analysis. The mesopore volume is 0.067 cm³/g. The NL-DFT pore size distribution curve reveals mesopore size ranges from 18-46 nm.

### Example 5—CHA: SSZ-13 Zeolite Monolith Preparation from an Adapted Method of Angew. Chem. Int. Ed. 2020, 59, Pages 23491-23495

1.08 g of sodium aluminate was dissolved in 8.8 g of distilled water under vigorous magnetic stirring to form a clear solution 1.

3.4 g of NaOH (97+%, ACS reagent, pellet, Acros Organics), 1.6 g of KOH (85%, pellet, Alfa Aesar), and 0.47 g of CsOH (50 wt % in H₂O, Sigma-Aldrich) were added to solution 1 under vigorous magnetic stirring. 20 g of LUDOX® AS-40 colloidal silica (40 wt. % suspension in H₂O, Sigma-Aldrich) was added dropwise into solution 1 under vigorous magnetic stirring for 2 h. The synthesis mixture was aged static in a Schott bottle at room temperature for 13 days. The mixture was then stirred for 3 days and thermally treated for 3 days in a preheated synthesis oven at 90° C.

After the solvothermal synthesis, the SSZ-13 nanocrystals were purified by repeated centrifugation (5250×g) and re-dispersion in distilled water until pH 7-8. The gel was dried at 25° C. for 3 days to obtain the monolithic structure. The monolith was further dried at 50° C. for 3 days and calcined at 550° C. in a muffle furnace for 6 h with a ramp rate of 1° C./min.

The calcined SSZ-13 monolith was then transformed into the ammonium form by a two-fold ion exchange procedure with 0.2 M aqueous ammonium chloride solution under static conditions at 80° C. overnight. The NH₄—SSZ-13 monolith was dried at room temperature for 3 days and then at 50° C. for 3 days. NH₄—SSZ-13 monolith was calcined at 550° C. in a muffle furnace for 6 h with a ramp rate of 1° C./min to achieve the H-SSZ-13 form.

H-SSZ-13 zeolite monolith has a BET area of 346 m²/g. The calculated micropore volume is 0.11 cm³/g according to the t-plot analysis. The mesopore volume is 0.18 cm³/g. The NL-DFT pore size distribution curve reveals mesopore size ranges from 2-50 nm.

The Examples presented herein demonstrate the synthesis of zeolite body or bodies in accordance with the present invention. The exemplified zeolite bodies had no measurable macroporosity and higher envelope density than known zeolite bodies. The Examples demonstrate the success in the preparation of self-supported zeolite monoliths with ordered mesostructures that are formed via close-packing of nanocrystalline zeolites without applied pressures, binders or replicas.

Every document cited herein, including any cross-referenced or related patent or application, is hereby incorporated by reference in its entirety unless expressly excluded or otherwise limited.

It will be appreciated that various modifications may be made to the embodiments shown without departing from the spirit and scope of the invention as defined by the accompanying claims.

The invention may be further understood with reference to the following clauses which are non-limiting:
1. A mesoporous zeolite body or bodies,
   wherein the or each zeolite body has a maximum internal diameter of 0.1 mm to 50 mm;
   wherein the or each zeolite body has an envelope density of between 0.7 g/cm³ and 1.4 g/cm³;
   and wherein macropores comprise less than 10% of the envelope volume of the or each zeolite body.
2. A mesoporous zeolite body or bodies according to clause 1, wherein the or each zeolite body has a maximum internal diameter of from about 0.5 mm to about 25 mm, preferably from about 2 mm to about 6 mm.
3. The or each zeolite body according to clause 1 or 2 having a micropore volume of from about 0.1 cm³ g⁻¹ to about 0.3 cm³ g⁻¹.
4. The or each zeolite body according to clause 1 or clause 2 or clause 3 having a mesopore volume of from about 0.1 cm³ g⁻¹ to about 0.8 cm³ g⁻¹.
5. The or each zeolite body according to any preceding clause having a Brunauer-Emmet-Teller (BET) area of from about 100 m² g⁻¹ to about 900 m² g⁻¹.
6. The or each zeolite body according to any preceding clause being an aluminosilicate zeolite body, preferably wherein the aluminosilicate zeolite has the chemical formula NaₙAlₙSi₉₆₋ₙO₁₉₂·16H₂O (0<n<27).
7. The or each zeolite body according to any preceding clause consisting essentially of nanocrystalline zeolite and/or consisting essentially of a single zeolite.
8. A method of preparing one or more zeolite bodies as defined in clauses 1 to 7, wherein the method comprises the steps of:
   a. mixing two or more zeolite precursors to form an organic template-containing synthesis solution;
   b. heating the synthesis solution to obtain a nanocrystalline zeolite colloidal suspension;
   c. concentrating the nanocrystalline zeolite colloidal suspension by centrifugation to obtain a wet nanocrystalline zeolite body; and
   d. drying the wet nanocrystalline zeolite body to form one or more of said zeolite bodies; and
   e. removing organic template to obtain one or more substantially template-free zeolite bodies.
9. A method of preparing one or more zeolite bodies having an envelope density of greater than about 0.7 g/cm³, the method comprising the steps of:
   f. mixing two or more zeolite precursors to form an organic template-containing synthesis solution;
   g. heating the synthesis solution to a sufficient temperature for a sufficient time to obtain a nanocrystalline zeolite colloidal suspension;
   h. concentrating the nanocrystalline zeolite colloidal suspension by centrifugation to obtain a wet nanocrystalline zeolite body;
   i. drying the wet nanocrystalline zeolite body to form one or more solid organic template-containing zeolite bodies, preferably having a maximum internal diameter of 0.1 mm to 50 mm; and
   j. heating the one or more organic template-containing zeolite bodies to remove the organic template and obtain one or more substantially template-free zeolite bodies.
10. The method according to clause 8 or 9 further comprising the step of f transforming the one or more substantially template-free zeolite bodies, preferably to its ammonium form, by an ion exchange method, preferably followed by drying and optionally further calcining the one or more zeolite bodies to remove ammonium ions.
11. The method according to clause 8 or 9 or 10 wherein step b) is a heating process comprising i) heating the solution to a temperature of from about 65° C. to about 75° C. for from about 2 to about 7 days and/or ii) subsequently heating the solution to a temperature of from about 45° C. to about 60° C. for a further from about 2 to about 7 days.
12. The method according to any one of clauses 8 to 11 wherein step d) is a drying process comprising i) drying the wet nanocrystalline zeolite body at approximately room temperature for about 6 hours to about 5 days and/or ii) subsequently heating the dried zeolite body to from about 45° C. to about 60° C. for from about 6 hours to about 5 days.
13. The method according to any one of clauses 8 to 12 wherein the precursors include tetrapropylammonium aluminate, and tetraethyl orthosilicate hydrolysed with tetrapropylammonium hydroxide.
14. A zeolite body or bodies manufactured according to the methods according to clauses 8 to 13.
15. The use of a zeolite body or bodies according to clauses 1 to 7 and 14 or a zeolite body or bodies prepared according to a method according to clauses 8 to 13 in catalysis or adsorption.

1.-21. (canceled)

22. A mesoporous zeolite body or bodies comprising greater than 85% zeolite by weight of the or each zeolite body,
wherein the or each zeolite body has a maximum internal diameter of 0.25 mm to 50 mm;
wherein the or each zeolite body has an envelope density of between 0.7 g/cm³ and 1.8 g/cm³;
and wherein macropores comprise less than 10% of an envelope volume of the or each zeolite body.

23. The mesoporous zeolite body or bodies according to claim 22 wherein the zeolite forming the or each zeolite body is nanocrystalline zeolite having a mean particle size of less than 1000 nm.

24. A mesoporous zeolite body or bodies comprising greater than 85% zeolite by weight of the or each zeolite body,
wherein the or each zeolite body has a maximum internal diameter of 0.25 mm to 50 mm;
wherein the or each zeolite body has an envelope density of between 0.7 g/cm³ and 1.8 g/cm³;
wherein the zeolite forming the or each zeolite body is nanocrystalline zeolite having a mean particle size of less than 1000 nm.

25. The mesoporous zeolite body or bodies according to claim 24 wherein the or each zeolite body has a maximum internal diameter of from about 0.5 mm to about 25 mm.

26. The mesoporous zeolite body or bodies according to claim 24 comprising greater than 95% zeolite by weight of the or each zeolite body.

27. The mesoporous zeolite body or bodies according to claim 24 having a micropore volume of from about 0.1 cm³ g⁻¹ to about 0.4 cm³ g⁻¹.

28. The mesoporous zeolite body or bodies according to claim 24 having a mesopore volume of from about 0.1 cm³ g⁻¹ to about 0.8 cm³ g⁻¹.

29. The mesoporous zeolite body or bodies according to claim 24 having a Brunauer-Emmet-Teller (BET) area of from about 100 m² g⁻¹ to about 900 m² g⁻¹.

30. The mesoporous zeolite body or bodies according to claim 24 being an aluminosilicate zeolite body, wherein the aluminosilicate zeolite has a chemical formula M_{2/n}OAl₂O₃·xSiO₂·yH₂O, where a charge-balancing non-framework cation M has valence n, x is 2.0 or more, and y represents moles of water in voids.

31. The mesoporous zeolite body or bodies according to claim 24 consisting essentially of nanocrystalline zeolite and/or consisting essentially of a single zeolite.

32. The mesoporous zeolite body or bodies according to claim 24, wherein the or each zeolite body further comprises either as part of the zeolite framework via ion exchange or impregnation with cations, or as metal oxides, one or more heteroatoms selected from the group consisting of: Cu, Ag, Mg, Ca, Sr, Ti, Zr, Hf, Zn, Cd, B, Al, Ga, Sn, Pb, Pt, Pd, Re, Rh, V, P, Zn, Sb, Rb, Li, Cs, Ag, Ba, Cr, Mo, W, Mn, Re, Fe, Co, Ni, Ge and noble metals.

33. A method of preparing one or more zeolite bodies, wherein the method comprises the steps of:
a. mixing two or more zeolite precursors to form an organic template-containing synthesis solution;
b. heating the synthesis solution to obtain a nanocrystalline zeolite colloidal suspension;
c. concentrating the nanocrystalline zeolite colloidal suspension by centrifugation to obtain a wet nanocrystalline zeolite body;
d. drying the wet nanocrystalline zeolite body at less than 50° C. to form one or more of said zeolite bodies; and
e. removing organic template to obtain one or more substantially template-free zeolite bodies.

34. A method of preparing one or more zeolite bodies having an envelope density of greater than about 0.7 g/cm³, the method comprising the steps of:
a. mixing two or more zeolite precursors to form an organic template-containing synthesis solution;
b. heating the synthesis solution to a sufficient temperature for a sufficient time to obtain a nanocrystalline zeolite colloidal suspension;
c. concentrating the nanocrystalline zeolite colloidal suspension by centrifugation to obtain a wet nanocrystalline zeolite body;
d. drying the wet nanocrystalline zeolite body at less than 50° C. to form one or more solid organic template-containing zeolite bodies having a maximum internal diameter of 0.1 mm to 50 mm; and
e. heating the one or more organic template-containing zeolite bodies to remove the organic template and obtain one or more substantially template-free zeolite bodies.

35. The method according to claim 34 further comprising the step of transforming the one or more substantially template-free zeolite bodies to ammonium form, by an ion exchange method, followed by drying and calcining the one or more zeolite bodies to remove ammonium ions.

36. The method according to claim 34 wherein step b) is a heating process comprising i) heating the solution to a temperature of from about 50° C. to about 100° C. for from about 2 to about 7 days and/or ii) subsequently heating the solution to a temperature of from about 45° C. to about 70° C. for from about 2 to about 7 days.

37. The method according to claim 34 wherein step d) is a drying process comprising drying the wet nanocrystalline zeolite body at about 20 C to about 50° C. for about 6 hours to about 5 days.

38. The method according to claim 34 wherein step d) is not carried out under vacuum.

39. The method according to claim 34, wherein the precursors include tetrapropylammonium-aluminate solution, and tetraethyl orthosilicate hydrolysed with tetrapropylammonium hydroxide.

40. A zeolite body or zeolite bodies manufactured according to the method of claim 34.

41. A plurality of zeolite bodies according to claim 22 wherein the zeolite bodies have a bulk density of greater than 0.6 g/cm³.
